(19) United States
(12) Patent Application Publication
(10) Pub. No.: US 2011/0160227 A1
(43) Pub. Date: Jun. 30, 2011

(54) Prolyl Hydroxylase Inhibitors

(76) Inventors: Antony Shaw, Collegeville, PA (US); Rosanna Tedesco, Collegeville, PA (US)

(21) Appl. No.: 13/059,478

(22) PCT Filed: Aug. 21, 2009
(86) PCT No.: PCT/US2009/054573

§ 371 Date: Feb. 17, 2011

Related U.S. Application Data
(60) Provisional application No. 61/090,699, filed on Aug. 21, 2008.

(51) Int. Cl. A61K 31/519; C07D 471/04; A61P 7/06

(52) U.S. Cl. 514/259.4; 544/282

(57) ABSTRACT

The invention described herein relates to certain bicyclic heteroaromatic N-substituted glycine derivatives of formula (I)

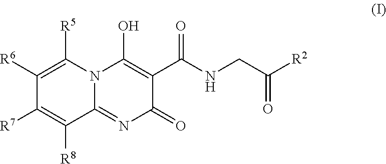

which are antagonists of HIF prolyl hydroxylases and are useful for treating diseases benefiting from the inhibition of this enzyme, anemia being one example.

### FIELD OF THE INVENTION

This invention relates to certain bicyclic heteroaromatic N-substituted glycine derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1 alpha, HIF-2alpha, and HIF-3alpha) are rapidly degraded by proteosome under normoxic conditions upon hydroxylation of proline residues by prolyl hydroxylases (EGLN1, 2, 3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also benefit by administering these compounds.

### SUMMARY OF THE INVENTION

In the first instance, this invention relates to a compound of formula (I):

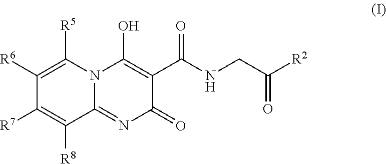

wherein:

R² is —NR³R⁴ or —OR⁹;

R³ and R⁴ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;

R⁵, R⁶, R⁷ and R⁸ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CON R¹⁰R¹¹, —N(R¹⁰)C(O)R¹², —N(R¹⁰)C(O)OR¹², —OC(O)N R¹⁰R¹¹, —N(R¹⁰)C(O)N R¹⁰R¹¹, —P(O)(OR¹²)₂, —SO₂N R¹⁰R¹¹, —N(R¹⁰)SO₂R¹², C₁-C₁₀ alkyl, C₁-C₁₀ alkenyl, C₁-C₁₀ alkynyl, C₃-C₆ cycloalkyl, C₃-C₆ heterocycloalkyl, aryl and heteroaryl group;

R⁹ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;

R¹⁰ and R¹¹ are each independently selected from the group consisting of hydrogen, C₁-C₁₀ alkyl, C₃-C₈cycloalkyl, C₁C₁₀ alkyl-C₃-C₈cycloalkyl, C₃-C₈heterocycloalkyl, C₁C₁₀ alkyl-C₃-C₈heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;

each R¹² is independently selected from the group consisting of hydrogen, C₁-C₁₀alkyl, C₂-C₁₀alkenyl, C₂-C₁₀alkynyl, C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;

any carbon or heteroatom of R², R³, R⁴, R⁵, R⁶, R⁷, R⁸, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —NR¹⁰R¹¹, CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₁-C₁₀ alkenyl, C₁-C₁₀ alkynyl, C₃-C₆ cycloalkyl, C₃-C₆ heterocycloalkyl, aryl or heteroaryl group, wherein R¹⁰, R¹¹, and R¹² are the same as defined above;

or a pharmaceutically acceptable salt or solvate thereof.

In a second aspect of the present invention, there is provided a compound of formula (I) or a salt or solvate thereof for use in mammalian therapy, e.g. treating anemia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁-C₄ alkyl” and “C₁-C₁₀alkyl” refers to an alkyl group having at least 1 and up to 4 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃-C₈ cycloalkyl” refers to a non-aromatic cyclic hydrocarbon ring having from three to eight carbon atoms. Exemplary “C₃-C₈ cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hückel's Rule, has the specified number of ring atoms, and that ring contains at least one heteratom selected from N, O, and/or S. Examples of “heteroaryl” groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl.

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids amd pharmaceutically-acceptable organic acids. Representative pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compounds of particular interest include those wherein:

R² is —OR⁹;

R⁵, R⁶, R⁷ and R⁸ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CON R¹⁰R¹¹, —N(R¹⁰)C(O)R¹², —N(R¹⁰)C(O)OR¹², —OC(O)N R¹⁰R¹¹, —N(R¹⁰)C(O)N R¹⁰R¹¹, —P(O)(OR¹²)₂, —SO₂N R¹⁰R¹¹, —N(R¹⁰)SO₂R¹², C₁-C₁₀ alkyl, C₁-C₁₀ alkenyl, C₁-C₁₀ alkynyl, C₃-C₆ cycloalkyl, C₃-C₆ heterocycloalkyl, aryl and heteroaryl group;

R⁹ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;

R¹⁰ and R¹¹ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;

each R¹² is independently selected from the group consisting of hydrogen, C₁-C₁₀alkyl, C₂-C₁₀alkenyl, C₂-C₁₀alkynyl, C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;

any carbon or heteroatom of R², R³, R⁴, R⁵, R⁶, R⁷, R⁸, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₁-C₁₀ alkenyl, C₁-C₁₀ alkynyl, C₃-C₆ cycloalkyl, C₃-C₆ heterocycloalkyl, aryl or heteroaryl group, wherein R¹⁰, R¹¹, and R¹² are the same as defined above;

or a pharmaceutically acceptable salt or solvate thereof.

Compounds of further interest are those wherein:

R² is —OR⁹;

R⁵, R⁶, R⁷ and R⁸ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CON R¹⁰R¹¹, —N(R¹⁰)C(O)R¹², —N(R¹⁰)C(O)OR¹², —OC(O)N R¹⁰R¹¹, —N(R¹⁰)C(O)N R¹⁰R¹¹, —P(O)(OR¹²)₂, —SO₂N R¹⁰R¹¹, —N(R¹⁰)SO₂R¹², C₁-C₁₀ alkyl, C₁-C₁₀ alkenyl, C₁-C₁₀ alkynyl, C₃-C₆ cycloalkyl, C₃-C₆ heterocycloalkyl, aryl and heteroaryl group;

R⁹ is H or a cation;

R¹⁰ and R¹¹ are each independently selected from the group consisting of hydrogen, C₁-C₁₀ alkyl, C₃-C₈cycloalkyl, C₁C₁₀ alkyl-C₃-C₈cycloalkyl, C₃-C₈heterocycloalkyl, C₁-C₁₀ alkyl-C₃-C₈heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;

each R¹² is independently selected from the group consisting of hydrogen, C₁-C₁₀alkyl, C₂-C₁₀alkenyl, C₂-C₁₀alkynyl, C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;

any carbon or heteroatom of R², R³, R⁴, R⁵, R⁶, R⁷, R⁸, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —NR¹⁰R¹¹, CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₁-C₁₀ alkenyl, C₁-C₁₀ alkynyl, C₃-C₆ cycloalkyl, C₃-C₆ heterocycloalkyl, aryl or heteroaryl group, wherein R¹⁰, R¹¹, and R¹² are the same as defined above;

or a pharmaceutically acceptable salt or solvate thereof.

Of further interest are those compounds which are:

N-[(4-hydroxy-2-oxo-2H-pyrido[1,2-a]pyrimidin-3-yl)carbonyl]glycine,
N-({4-hydroxy-9-[(1-methylethyl)oxy]-2-oxo-2H-pyrido[1,2-a]pyrimidin-3-yl}carbonyl)glycine,
N-[(7 -bromo-4-hydroxy-2-oxo-2H-pyrido [1,2-a]pyrimidin-3-yl)carbonyl]glycine,
N-({4-hydroxy-7-[(1-methylethyl)oxy]-2-oxo-2H-pyrido[1,2-a]pyrimidin-3-yl}carbonyl)glycine.

Processes for preparing the compound of formula (I) are also within the ambit of this invention. To illustrate, one process for preparing a compound of formula (I)

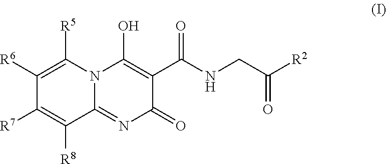

wherein R², R⁵, R⁶, R⁷ and R⁸ are the same as defined above for formula (I), comprising treating a compound of formula A:

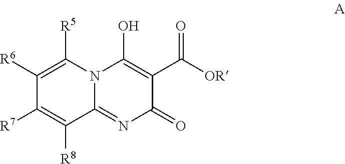

wherein R⁵, R⁶, R⁷ and R⁸ are the same as for those groups in formula (I) and R′ is an ester-forming group, with glycine sodium salt or glycine and an appropriate base, such as 1,8-diazabicyclo[5.4.0]undec-7-ene, sodium ethoxide or sodium hydride, in an appropriate solvent, such as ethanol or 2-methoxyethanol, under either conventional thermal conditions or by microwave irradiation, to form a compound of formula (I) where R² is —OH. A second process for preparing a compound of formula (I) wherein R², R⁵, R⁶, R⁷ and R⁸ are the same as defined above for formula (I), comprising treating a compound of formula B:

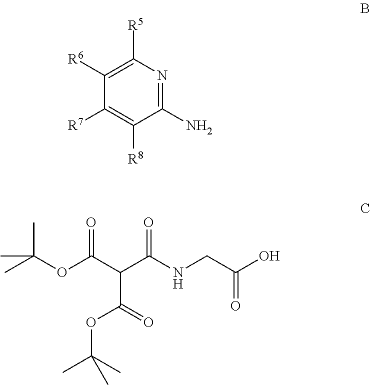

wherein R⁵, R⁶, R⁷ and R⁸ are the same as for those groups in formula (I), with the compound of formula C, N-(3-[(1,1-dimethylethyl)oxy]-2-{[(1,1-dimethylethyl)oxy]carbonyl}-3-oxopropanoyl)glycine, in an appropriate solvent, such as 1,2-dichlorobenzene, under either conventional thermal conditions or by microwave irradiation, to form a compound of formula (I) where R² is —OH.

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### Definitions:

DMSO—dimethylsulfoxide,
DBU—1,8-diazabicyclo[5.4.0]undec-7-ene,
ODS—octadecylsilane,
rp-HPLC—reverse-phase high performance liquid chromatography,
TFA—Trifluoroacetic acid.

### Chemical Background:

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

The compounds described herein may be made from commercially available starting materials or synthesized using known organic, inorganic and/or enzymatic processes.

### Illustrated Methods of Preparation

### Schemes

Included in the present invention are processes according to Schemes 1-2 for the synthesis of the compounds:

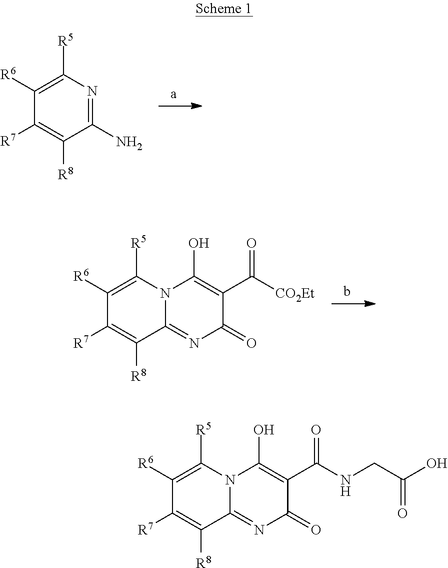

a) (EtO₂C)₃CH, PhMe or 1,2-Cl₂C₆H₄, 155-200° C., microwave; b) glycine, DBU, EtOH, 160° C., microwave or glycine, Na salt, MeO(CH₂)₂OH, reflux.

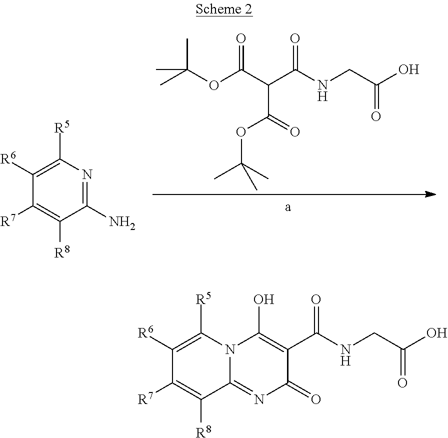

a) 1,2-Cl₂C₆H₄, 200° C., microwave.

### Example 1

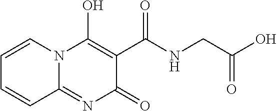

N-[(4-hydroxy-2-oxo-2H-pyrido[1,2-a]pyrimidin-3-yl)carbonyl]glycine

A mixture of 2-aminopyridine (0.100 g, 1.06 mmol), triethyl methanetricarboxylate (0.490 mL, 2.33 mmol) and toluene (2 mL) was stirred in a microwave reactor at 155° C. for 25 min. After cooling, ether (˜2 mL) was added and the precipitate filtered. A mixture of this precipitate, glycine (0.278 g, 3.71 mmol), 1,8-diazabicyclo[5.4.0]undec-7-ene (0.555 mL, 3.71 mmol) and ethanol (1.5 mL) was stirred in a microwave reactor at 160° C. for 25 min. After cooling, the mixture was diluted with water (5 mL), washed with ethyl acetate, acidified to pH 3-4 with 6 M aqueous hydrochloric acid and extracted with ethyl acetate. The extracts were washed with brine, then evaporated under reduced pressure. The residue was purified by preparative rp-HPLC (ODS, 10-90% acetonitrile/water+0.1% trifluoroacetic acid) to give the title compound (0.032 g, 11%) as a white powder. 1H NMR (400 MHz, DMSO-d₆) δ ppm 4.13 (d, J=5.56 Hz, 2 H) 7.40 (td, J=6.95, 1.26 Hz, 1 H) 7.56 (d, J=8.59 Hz, 1 H) 8.11 (ddd, J=8.72, 6.95, 1.77 Hz, 1 H) 8.95-9.00 (m, 1 H) 9.82 (t, J=5.18 Hz, 1 H) 12.93 (br. s., 1 H) 15.73 (br. s., 1 H).

### Example 2

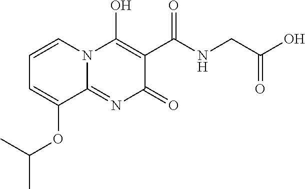

2a) N-({4-hydroxy-9-[(1-methylethyl)oxy]-2-oxo-2H-pyrido[1,2-a]pyrimidin-3-yl}carbonyl)glycine

Ethyl 4-hydroxy-9-[(1-methylethyl)oxy]-2-oxo-2H-pyrido[1,2-a]pyrimidine-3-carboxylate. A solution of 3-[(1-methylethyl)oxy]-2-pyridinamine (0.100 g, 0.657 mmol) and triethyl methanetricarboxylate (0.305 g, 1.31 mmol) in 1,2-dichlorobenzene (1 mL) was heated in a microwave reactor at 200° C. for 20 min, then cooled and chromatographed (silica gel, 1-9% methanol/dichloromethane) to give the title compound (0.118 g, 61%) as a gum, containing two tautomers. Major tautomer (˜87%): 1H NMR (400 MHz, DMSO-d₆) δ ppm 1.25 (t, J=7.07 Hz, 3 H) 1.36 (d, J=5.81 Hz, 6 H) 4.18 (q, J=7.07 Hz, 2 H) 4.86 (sept, J=6.06 Hz, 1 H) 7.29 (t, J=7.45 Hz, 1 H) 7.71 (d, J=7.83 Hz, 1 H) 8.52 (dd, J=6.95, 1.14 Hz, 1 H) 11.98 (br. s., 1 H). Minor tautomer (˜13%): 1H NMR (400 MHz, DMSO-d₆) δ ppm 1.21 (t, J=7.07 Hz, 3 H) 1.27 (d, J=5.81 Hz, 6 H) 4.06 (q, J=7.07 Hz, 1 H) 4.63 (sept, J=6.08 Hz, 1 H) 7.15 (dd, J=8.08, 4.80 Hz, 1 H) 7.43 (dd, J=8.21, 1.39 Hz, 1 H) 7.89 (dd, J=4.80, 1.52 Hz, 1 H) 8.90 (s, 1 H).

2b) N-({4-hydroxy-9-[(1-methylethyl)oxy]-2-oxo-2H-pyrido[1,2-a]pyrimidin-3-yl}carbonyl)glycine.

Glycine, sodium salt (0.116 g, 1.19 mmol), was added to a stirred solution of ethyl 2-hydroxy-9-[(1-methylethyl)oxy]-4-oxo-4H-pyrido[1,2-a]pyrimidine-3-carboxylate (0.116 g, 0.397 mmol) in 2-methoxyethanol (5 mL) and the mixture stirred for 2 h at reflux under nitrogen. After cooling overnight, water (30 mL) was added, followed by 6 M aqueous hydrochloric acid to pH 2. The mixture was stirred 0.5 h, then the precipitate filtered, washed with water and dried to give the title compound (0.076 g, 60%) as a cream solid. 1H NMR (400 MHz, DMSO-d₆) δ ppm 1.37 (d, J=6.06 Hz, 6 H) 4.13 (d, J=5.56 Hz, 2 H) 4.82 (sept, J=6.00 Hz, 1 H) 7.29 (dd, J=7.83, 7.07 Hz, 1 H) 7.56 (d, J=7.33 Hz, 1 H) 8.56 (dd, J=7.07, 1.26 Hz, 1 H) 9.83 (t, J=5.43 Hz, 1 H) 12.94 (br. s., 1 H) 15.62 (s, 1 H).

### Example 3

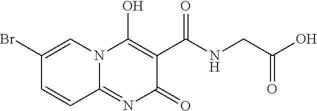

3a) N-[(7-bromo-4-hydroxy-2-oxo-2H-pyrido[1,2-a]pyrimidin-3-yl)carbonyl]glycine

N-(3-[(1,1-Dimethylethyl)oxy]-2-{[(1,1-dimethylethyl)oxy]carbonyl}-3-oxopropanoyl)glycine. Sodium hydride (0.407 g of a 60% oil suspension, 10.2 mmol) was added to an ice-cooled, stirred solution of di-tert-butyl malonate (2.00 g, 9.25 mmol) in THF (30 mL) under nitrogen. The mixture was warmed to room temperature and stirred 15 min, giving a colourless solution. Ethyl isocyanatoacetate (1.14 mL, 10.2 mmol) was injected into the mixture and the that mixture stirred for 5 min at room temperature and 1 h under reflux, then cooled and poured into ice-cold/0.1 M aqueous hydrochloric acid (130 mL). The mixture was extracted with ethyl acetate and the extracts washed with water, brine, then dried (MgSO₄) and the solvent evaporated under reduced pressure. The residue was chromatographed (silica gel, 0-9% methanol/dichloromethane) to give the intermediate ester (1.54 g). 1 M aqueous sodium hydroxide (22 mL) was added dropwise to an ice-cooled, stirred solution of the intermediate in ethanol (90 mL). After stirring 4 h, the mixture was concentrated to about 25 mL under reduced pressure, diluted with water (100 mL), filtered and acidified to pH 2 with 1 M aqueous hydrochloric acid with ice cooling and extracted with ethyl acetate. The extracts were washed with water, brine, dried (MgSO₄) and the solvent evaporated under reduced pressure. The residue was chromatographed (silica gel, 1-9% methanol/dichloromethane+0.5% acetic acid) to give the title compound (0.722 g, 25%) as a white solid. 1H NMR (400 MHz, DMSO-d₆) δ ppm 1.41 (s, 18 H) 3.82 (d, J=5.56 Hz, 2 H) 4.31 (s, 1 H) 8.40 (t, J=5.56 Hz, 1 H) 12.69 (br. s., 1 H).

3b) N-[(7-bromo-4-hydroxy-2-oxo-2H-pyrido[1,2-a]pyrimidin-3-yl)carbonyl]glycine.

A mixture of 2-amino-5-bromopyridine (0.120 g, 0.694 mmol), N-(3-[(1,1-dimethylethyl)oxy]-2-{[(1,1-dimethylethyl)oxy]carbonyl}-3-oxopropanoyl)glycine (0.220 g, 0.694 mmol) and 1,2-dichlorobenzene (1.5 mL) was heated in a microwave reactor at 200° C. for 20 min, then cooled. The precipitate was filtered, washed with ether and dried. The solid was dissolved in 1 M aqueous sodium hydroxide (5 mL), methanol (15 mL) and water (60 mL), then the solution was filtered and re-acidified to pH 1 slowly with 6 M aqueous hydrochloric acid. The precipitate was filtered, washed with water and dried to give the title compound (0.116 g, 49%) as a light brown solid. 1H NMR (400 MHz, DMSO-d₆) δ ppm 4.13 (d, J=5.56 Hz, 2 H) 7.52 (dd, J=9.35, 0.51 Hz, 1 H) 8.23 (dd, J=9.35, 2.27 Hz, 1 H) 9.00 (dd, J=2.27, 0.76 Hz, 1 H) 9.75 (t, J=5.43 Hz, 1 H) 12.94 (br. s., 1 H) 15.84 (br. s., 1 H).

### Example 4

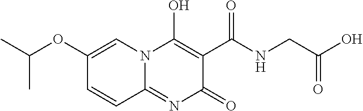

4a) N-({4-hydroxy-7-[(1-methylethyl)oxy]-2-oxo-2H-pyrido[1,2-a]pyrimidin-3-yl}carbonyl)glycine-5-[(1-methylethyl)oxy]-2-pyridinamine.

A mixture of 5-bromo-2-pyridinamine (1.00 g, 5.78 mmol), 2,5-hexanedione (0.792 g, 6.94 mmol), p-toluenesulfonic acid monohydrate (0.110 g, 0.578 mmol) and toluene (6 mL) was heated in a microwave reactor at 150° C. for 0.5 h, then cooled and diluted with ethyl acetate (50 mL). The mixture was washed with saturated sodium bicarbonate, dried (MgSO₄) and the solvent evaporated under reduced pressure to leave the crude pyrrole as a brown oil. Sodium hydride (0.694 g of a 60% oil suspension, 17.3 mmol) was added in portions to an ice-cooled, stirred solution of the crude pyrrole in N,N-dimethylformamide (3 mL) and 2-propanol (3 mL) under nitrogen. After the mixture had stopped bubbling, copper (I) iodide (0.165 g, 0.867 mmol) was added. The mixture was heated in a microwave reactor at 120° C. for 0.5 h, then cooled, poured into saturated aqueous ammonium chloride (60 mL) and extracted with ethyl acetate. The extracts were washed with 5% aqueous ammonia, brine, then dried (MgSO₄) and the solvent evaporated under reduced pressure to leave a brown oil, sufficiently pure for the next step. A mixture of this brown oil, hydroxylamine hydrochloride (2.61 g, 37.6 mmol), triethylamine (1.61 mL, 11.6 mmol) and 2:1 ethanol/water (12 mL) was stirred under reflux under nitrogen for 18 h, then cooled and poured into 1 M aqueous hydrochloric acid (60 mL). The mixture was washed with ether, adjusted to pH 10 (6 M aqueous sodium hydroxide) and extracted with dichloromethane. The extracts were dried (Na₂SO₄) and the dichloromethane evaporated under reduced pressure. The residue was chromatographed (silica gel, 1-9% methanol/dichloromethane) to give the title compound (0.448 g, 51%) as an oil. 1H NMR (400 MHz, DMSO-d₆) δ ppm 1.19 (d, J=6.06 Hz, 6 H) 4.32 (sept, J=6.06 Hz, 1 H) 5.46 (br. s., 2 H) 6.40 (d, J=8.84 Hz, 1 H) 7.09 (dd, J=8.84, 3.03 Hz, 1 H) 7.60 (d, J=2.27 Hz, 1 H).

4b) N-({4-hydroxy-7-[(1-methylethyl)oxy]-2-oxo-2H-pyrido[1,2-a]pyrimidin-3-yl}carbonyl)glycine.

A mixture of 5-[(1-methylethyl)oxy]-2-pyridinamine (0.074 g, 0.486 mmol), N-(3-[(1,1-dimethylethyl)oxy]-2-{[(1,1-dimethylethyl)oxy]carbonyl}-3-oxopropanoyl)glycine (0.130 g, 0.410 mmol) and 1,2-dichlorobenzene (2 mL) was heated in a microwave reactor at 200° C. for 0.5 h, then cooled and poured into 0.1 M aqueous sodium hydroxide (70 mL). The mixture was washed with ether, then acidified to pH 1 with 6 M aqueous hydrochloric acid and extracted with ethyl acetate. The extracts were washed with brine, dried (MgSO₄) and the solvent evaporated under reduced pressure. The residue was purified by reverse-phase preparative HPLC (ODS, 10-90% acetonitrile/water+0.1% trifluoroacetic acid) to give the title compound (0.044 g, 33%) as a solid. 1H NMR (400 MHz, DMSO-d₆) δ ppm 1.35 (d, J=6.06 Hz, 6 H) 4.14 (d, J=5.56 Hz, 2 H) 4.75 (sept, J=6.06 Hz, 1 H) 7.55 (d, J=9.60 Hz, 1 H) 7.94 (dd, J=9.60, 2.78 Hz, 1 H) 8.44 (d, J=2.78 Hz, 1 H) 9.88 (t, J=5.18 Hz, 1 H) 12.95 (br. s., 1 H) 15.47 (br. s., 1 H).

### Biological Background:

The following references set out information about the target enzymes, HIF prolyl hydroxylases, and methods and materials for measuring inhibition of same by small molecules.

M. Hirsilä, P. Koivunen, V. Günzler, K. I. Kivirikko, and J. Myllyharju “Characterization of the Human Prolyl 4-Hydroxylases That Modify the Hypoxia-inducible Factor” J. Biol. Chem., 2003, 278, 30772-30780.

C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor α” Advan. Enzyme Regul., 2004, 44, 75-92

M. S. Wiesener, J. S. Jürgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2α in distinct cell populations of different organs” FASEB J., 2003, 17, 271-273.

S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFα) stabilizers for enhancing erythropoiesis” PCT Int. Appl. (2004), WO 2004108121 A1

C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.

### For the Expression of EGLN3 See:

R. K. Bruick and S. L. McKnight “A Conserved Family of Prolyl-4-Hydroxylases That Modify HIF” Science, 2001, 294, 1337-1340.

### For the Expression of HIF2α-CODD See:

a) P. Jaakkola, D. R. Mole, Y.-M. Tian, M. I. Wilson, J. Gielbert, S. J. Gaskell, A. von Kriegsheim, H. F. Hebestreit, M. Mukherji, C. J. Schofield, P. H. Maxwell, C. W. Pugh, P, J. Ratcliffe “Targeting of HIF-α to the von Hippel-Lindau Ubiquitylation Complex by O₂-Regulated Prolyl Hydroxylation” Science, 2001, 292, 468-472.

b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂ Sensing” Science, 2001, 292, 464-468.

### For the Expression of VHL, Elongin b and Eelongin c See:

A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### Biological Assay(s)

### EGLN3 Assay

### Materials:

His-MBP-EGLN3 (6HisMBPAttB1EGLN3(1-239)) was expressed in E. Coli and purified from an amylase affinity column. Biotin-VBC [6HisSumoCysVHL(2-213), 6HisSumoElonginB(1-118), and 6HisSumoElonginC(1-112)] and His-GB1-HIF2α-CODD (6HisGB1tevHIF2A(467-572)) were expressed from E. Coli.

### Method:

Cy5-labelled HIF2α CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 nL of inhibitors in DMSO (or DMSO controls) were stamped into a 384-well low volume Corning NBS plate, followed by addition of 2.5 μL of enzyme [50 mL buffer (50 mM HEPES/50 mM KCl)+1 mL of a 10 mg/mL BSA in buffer+6.25 μL of a 10 mg/mL FeCl₂ solution in water+100 μL of a 200 mM solution of ascorbic acid in water+15.63 μL EGLN3] or control [50 mL buffer+1 mL of a 10 mg/mL BSA in buffer+6.25 μL of a 10 mg/mL FeCl₂ solution in water+100 μL of a 200 mM solution of ascorbic acid in water]. Following a 3 minutes incubation, 2.5 μL of substrate [50 mL Buffer+68.6 μL biotin-VBC+70.4 μL Eu (at 710 μg/mL stock)+91.6 μL Cy5CODD+50 μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c)̂d), where a is the minimum % activity, b is the maximum % activity, c is the pIC₅₀, and d is the Hill slope.

The IC₅₀ for exemplified compounds in the EGLN3 assay ranged from approximately 3-80 nanomolar. This range represents the data accumulated as of the time of the filing of this initial application. Later testing may show variations in IC₅₀ data due to variations in reagents, conditions and variations in the method(s) used from those given herein above. So this range is to be viewed as illustrative, and not a absolute set of numbers.

### Measure Epo Protein Produced by Hep3B Cell Line Using ELISA Method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37degC/5% CO2/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

The EC₅₀ for exemplar compounds in the Hep3B ELISA assay ranged from approximately 28—greater than a 100 micromolar, the upper limit of the assay, using the reagents and under the conditions outlined herein above. This range represents the data accumulated as of the time of the filing of this initial application. Later testing may show variations in EC₅₀ data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permitted therapeutic regime.

The foregoing examples and assay have been set forth to illustrate the invention, not limit it. What is reserved to the inventors is to be determined by reference to the claims.

What is claimed is:

1. A compound of formula (I): 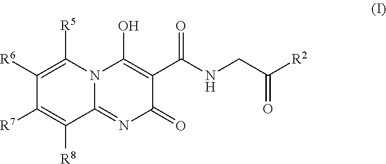
wherein:
R² is —NR³R⁴ or —OR⁹;
R³ and R⁴ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;
R⁵, R⁶, R⁷ and R⁸ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CON R¹⁰R¹¹, —N(R¹⁰)C(O)R¹², —N(R¹⁰)C(O)OR¹², —OC(O)N R¹⁰R¹¹, —N(R¹⁰)C(O)N R¹⁰R¹¹, —P(O)(OR¹²)₂, ——SO₂N R¹⁰R¹¹, —N(R¹⁰)SO₂R¹³, C₁-C₁₀ alkyl, C₁-C₁₀ alkenyl, C₁-C₁₀ alkynyl, C₃-C₆ cycloalkyl, C₃-C₆ heterocycloalkyl, aryl and heteroaryl group;
R⁹ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;
R¹⁰ and R¹¹ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;
each R¹² is independently selected from the group consisting of hydrogen, C₁-C₁₀alkyl, C₂-C₁₀alkenyl, C₂-C₁₀alkynyl, C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
any carbon or heteroatom of R², R³, R⁴, R⁵, R⁶, R⁷, R⁸, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₁-C₁₀ alkenyl, C₁-C₁₀ alkynyl, C₃-C₆ cycloalkyl, C₃-C₆ heterocycloalkyl, aryl or heteroaryl group, wherein R¹⁰, R¹¹, and R¹² are the same as defined above;
or a pharmaceutically acceptable salt or solvate thereof.

2. A compound according to claim 1 wherein:
R² is —OR⁹;
R⁵, R⁶, R⁷ and R⁸ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CON R¹⁰R¹¹, —N(R¹⁰)C(O)R¹², —N(R¹⁰)C(O)OR¹², —OC(O)N R¹⁰R¹¹, —N(R¹⁰)C(O)N R¹⁰R¹¹, —P(O)(OR¹²)₂, —SO₂N R¹⁰R¹¹, —N(R¹⁰)SO₂R¹², C₁-C₁₀ alkyl, C₁-C₁₀ alkenyl, C₁-C₁₀ alkynyl, C₃-C₆ cycloalkyl, C₃-C₆ heterocycloalkyl, aryl and heteroaryl group;
R⁹ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;
R¹⁰ and R¹¹ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;
each R¹² is independently selected from the group consisting of hydrogen, C₁-C₁₀alkyl, C₂-C₁₀alkenyl, C₂-C₁₀alkynyl, C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
any carbon or heteroatom of R², R³, R⁴, R⁵, R⁶, R⁷, R⁸, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R^{11,} —N(R¹⁰)SO₂R¹², C₁-C₁₀ alkenyl, C₁-C₁₀ alkynyl, C₃-C₆ cycloalkyl, C₃-C₆ heterocycloalkyl, aryl or heteroaryl group, wherein R¹⁰, R¹¹, and R¹² are the same as defined above;
or a pharmaceutically acceptable salt or solvate thereof.

3. A compound according to claim 1 wherein:
R² is —OR⁹;
R⁵, R⁶, R⁷ and R⁸ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CON R¹⁰R¹¹, —N(R¹⁰)C(O)R¹², —N(R¹⁰)C(O)OR¹², —OC(O)N R¹⁰R¹¹, —N(R¹⁰)C(O)N R¹⁰R¹¹, —P(O)(OR¹²)₂, —SO₂N R¹⁰R¹¹, —N(R¹⁰)SO₂R¹², C₁-C₁₀ alkyl, C₁-C₁₀ alkenyl, C₁-C₁₀ alkynyl, C₃-C₆ cycloalkyl, C₃-C₆ heterocycloalkyl, aryl and heteroaryl group;
R⁹ is H or a cation;
R¹⁰ and R¹¹ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;
each R¹² is independently selected from the group consisting of hydrogen, C₁-C₁₀alkyl, C₂-C₁₀alkenyl, C₂-C₁₀alkynyl, C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
any carbon or heteroatom of R², R³, R⁴, R⁵, R⁶, R⁷, R⁸, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₁-C₁₀ alkenyl, C₁-C₁₀ alkynyl, C₃-C₆ cycloalkyl, C₃-C₆ heterocycloalkyl, aryl or heteroaryl group, wherein R¹⁰, R¹¹, and R¹² are the same as defined above;
or a pharmaceutically acceptable salt or solvate thereof.

4. A compound according to claim 1 which is:
N-[(4-hydroxy-2-oxo-2H-pyrido[1,2-a]pyrimidin-3-yl)carbonyl]glycine,
N-({4-hydroxy-9-[(1-methylethyl)oxy]-2-oxo-2H-pyrido[1,2-a]pyrimidin-3-yl}carbonyl)glycine,
N-[(7-bromo-4-hydroxy-2-oxo-2H-pyrido[1,2-a]pyrimidin-3-yl)carbonyl]glycine,
N-({4-hydroxy-7-[(1-methylethyl)oxy]-2-oxo-2H-pyrido[1,2-a]pyrimidin-3-yl}carbonyl)glycine,
or a pharmaceutically acceptable salt or solvate thereof.

5. A method for treating anemia in a mammal, which method comprises administering an effective amount of a compound of formula (I) or a salt or solvate thereof according to claim 1 to a mammalian suffering from anemia which can be treated by inhibiting HIF prolyl hydroxylases.

6. A pharmaceutical composition comprising a compound of formula (I) or a salt, solvate, according to claim 1 and one or more of pharmaceutically acceptable carriers, diluents and excipients.

7. A process for preparing a compound of formula (I) 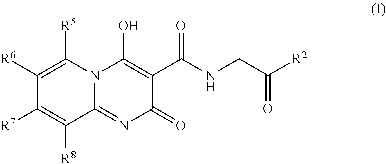
wherein:
R² is —NR³R⁴ or —OR⁹;
R³ and R⁴ are each independently selected from the group consisting of hydrogen, C₁-C₁₀ alkyl, C₂-C₁₀ alkenyl, C₂-C₁₀ alkynyl, C₃-C₈ cycloalkyl, C₃-C₈ heterocycloalkyl, aryl and heteroaryl;
R⁵, R⁶, R⁷ and R⁸ are each independently selected from the group consisting of hydrogen, nitro, cyano, halogen, —C(O)R¹², —C(O)OR¹², —OR¹², —SR¹², —S(O)R¹², —S(O)₂R¹², —NR¹⁰R¹¹, —CON R¹⁰R¹¹, —N(R¹⁰)C(O)R¹², —N(R¹⁰)C(O)OR¹², —OC(O)N R¹⁰R¹¹, —N(R¹⁰)C(O)N R¹⁰R¹¹, —P(O)(OR¹²)₂, —SO₂N R¹⁰R¹¹, —N(R¹⁰)SO₂R¹², C₁-C₁₀ alkyl, C₁-C₁₀ alkenyl, C₁-C₁₀ alkynyl, C₃-C₆ cycloalkyl, C₃-C₆ heterocycloalkyl, aryl and heteroaryl group;
R¹⁰ and R¹¹ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁-C₁₀alkyl-aryl, heteroaryl, C₁-C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), —SO₂(C₁-C₄ alkyl); or R¹⁰ and R¹¹ taken together with the nitrogen to which they are attached form a 5- or 6- or 7-membered saturated ring optionally containing one other heteroatom which is oxygen, nitrogen or sulphur;
each R¹² is independently selected from the group consisting of hydrogen, C₁-C₁₀alkyl, C₂-C₁₀alkenyl, C₂-C₁₀alkynyl, C₃-C₈ cycloalkyl, C₃-C₈heterocycloalkyl, C₆-C₁₄ aryl, C₁-C₁₀alkyl-aryl, heteroaryl, and C₁-C₁₀alkyl-heteroaryl;
R⁹ is H or a cation, or C₁-C₁₀alkyl which is unsubstituted or substituted with one or more substituents independently selected from the group consisting of C₃-C₆ cycloalkyl, heterocycloalkyl, aryl, and heteroaryl;
any carbon or heteroatom of R², R³, R⁴, R⁵, R⁶, R⁷, R⁸, R⁹, R¹⁰, R¹¹ or R¹² is unsubstituted or, where possible, is substituted with one or more substituents independently selected from C₁-C₆ alkyl, aryl, heteroaryl, halogen, —OR¹², —NR¹⁰R¹¹, cyano, nitro, —C(O)R¹², —C(O)OR¹², —SR¹², —S(O)R¹², —S(O)₂R¹², —NR¹⁰R¹¹, —CONR¹⁰R¹¹, —N(R¹⁰)C(O)R¹², —N(R¹⁰)C(O)OR¹², —OC(O)NR¹⁰R¹¹, —N(R¹⁰)C(O)NR¹⁰R¹¹, —SO₂NR¹⁰R¹¹, —N(R¹⁰)SO₂R¹², C₁-C₁₀ alkenyl, C₁-C₁₀ alkynyl, C₃-C₆ cycloalkyl, C₃-C₆ heterocycloalkyl, aryl or heteroaryl group, wherein R¹⁰, R¹¹, and R¹² are the same as defined above comprising treating a compound of formula A: 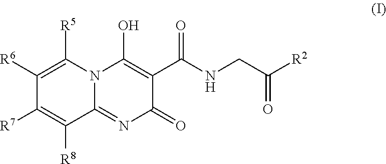
wherein R⁵, R⁶, R⁷ and R⁸ are the same as for those groups in formula (I) and R′ is an ester-forming group, with glycine sodium salt or glycine and an appropriate base, such as 1,8-diazabicyclo[5.4.0]undec-7-ene, sodium ethoxide or sodium hydride, in an appropriate solvent, such as ethanol or 2-methoxyethanol, under either conventional thermal conditions or by microwave irradiation, to form a compound of formula (I) where R² is —OH.
or a process for preparing a compound of formula (I) wherein R², R⁵, R⁶, R⁷ and R⁸ are the same as defined above for formula (I), comprising treating a compound of formula B: 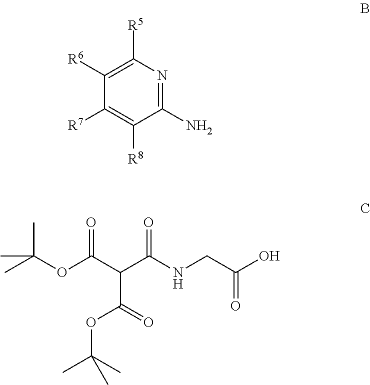
wherein R⁵, R⁶, R⁷ and R⁸ are the same as for those groups in formula (I), with the compound of formula C, N-(3-[(1,1-dimethylethyl)oxy]-2-{[(1,1-dimethylethyl)oxy]carbonyl}-3-oxopropanoyl)glycine, in an appropriate solvent, such as 1,2-dichlorobenzene, under either conventional thermal conditions or by microwave irradiation, to form a compound of formula (I) where R² is —OH.
